(19) United States
(12) Patent Application Publication
(10) Pub. No.: US 2014/0162966 A1
(43) Pub. Date: Jun. 12, 2014

(54) NANOPARTICLES FOR DELIVERY OF LIGANDS

(71) Applicant: The U.S.A, as represented by the Secretary, Department of Health & Human Services, Bethesda, MD (US)

(72) Inventors: Xiaoyuan Chen, Potomac, MD (US); Seulki Lee, Baltimore, MD (US); Ki Young Choi, Rockville, MD (US); Gang Liu, Xiamen (CN)

(21) Appl. No.: 14/086,111

(22) Filed: Nov. 21, 2013

Related U.S. Application Data
(60) Provisional application No. 61/729,159, filed on Nov. 21, 2012.

(51) Int. Cl. C07J 41/00

(52) U.S. Cl. CPC C07J 41/0061
USPC 514/26; 536/53; 536/23.1; 536/24.5

(57) ABSTRACT

Disclosed is a nanoparticulate complex comprising an artificial phosphate receptor of formula (I): P-[L-[-N(CH₂-2-pyridyl)₂]]ₚ.pZN²⁺ (I) wherein P represents a nanoparticulate substrate, L represents a linking group, and p is an integer of ≧1. Also disclosed are a method for silencing a gene in a cancer patient in need thereof, a method for treating or preventing cancer in a patient in need thereof, and a method for targeting a cell in cancer treatment comprising use of the nanoparticulate complex, for example, a DPA/Zn-functionalized nanoparticulate complex.

### CROSS-REFERENCE TO A RELATED APPLICATION

This application claims the benefit of U.S. Provisional Patent Application No. 61/729,159, filed Nov. 21, 2012, the disclosure of which is incorporated herein in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 2,490 Byte ASCII (Text) file named “715507 ST25.TXT,” created on Nov. 13, 2013.

### BACKGROUND OF THE INVENTION

Nuclei acid treatment has rapidly emerged as a potent therapeutic strategy. However, challenges remain in effectively delivering nucleic acids to target cells. For example, small interfering RNAs (siRNAs) silence gene expression in a highly specific manner for treating genetic disorders, signifying a new approach in cancer therapy through the regulation of aberrant gene expression inherent to cancer (Pecot C. V. et al., Nat. Rev. Cancer 2011; 11:59-67). However, the physicochemical characteristics of siRNA (i.e. high molecular weight, anionic charge, and hydrophilic character) hinder its passive diffusion across cell membranes precluding any therapeutic function (Whitehead K. A. et al., Nat. Rev. Drug Discov. 2008; 8:129-138). Furthermore, siRNA molecules are highly vulnerable to degradation. Thus, for effective siRNA delivery, siRNA carriers are needed to protect siRNA, facilitate cellular entry, avoid endosomal compartmentalization, and promote localization in the cytoplasm where the siRNA cargo can be recognized by the RNA-induced silencing complex (RISC).

Accordingly, there exists in the art a need for improved delivery systems for nucleic acids.

### BRIEF SUMMARY OF THE INVENTION

The invention provides a nanoparticulate complex, for example, a dipicolylamine (DPA)/Zn-functionalized nanoparticulate complex, comprising an artificial phosphate receptor of formula (I): P-[L-[-N(CH₂-2-pyridyl)₂]]ₚ.pZn²⁺ (I) wherein P represents a nanoparticulate substrate, L represents a linking group, and p is an integer of ≧1.

The invention also provides a phosphate anion ligand complex comprising at least one phosphate anion ligand complexed with a nanoparticulate complex.

The invention also provides an anticancer complex comprising an anticancer agent and a phosphate anion ligand complex.

The invention further provides a method for silencing a gene in a cancer patient in need thereof comprising administering an effective amount of a phosphate anion ligand complex comprising at least one phosphate anion ligand complexed with a nanoparticulate complex.

The invention additionally provides a method for treating or preventing cancer in a patient in need thereof, comprising administering an effective amount of a phosphate anion ligand complex comprising at least one phosphate anion ligand complexed with a nanoparticulate complex.

The invention also provides a method for targeting a cell in cancer treatment, comprising contacting the cell with a phosphate anion ligand complex comprising at least one phosphate anion ligand complexed with a nanoparticulate complex.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

FIG. 1 illustrates a synthesis of a dipicolylamine derivative 104 in accordance with an embodiment of the invention.

FIG. 2 illustrates a synthesis of an artificial phosphate receptor of formula (I): P-[L-[-N(CH₂-2-pyridyl)₂]ₚ].pZn²⁺ (106) in accordance with an embodiment of the invention.

FIG. 3 depicts the binding of siLuc by zinc-chelated dipicolylamine-functionalized hyaluronic acid nanoparticles (HA_{DPA-Zn}-NPs) but not by dipicolylamine-functionalized hyaluronic acid nanoparticles (HA_{DPA}-NPs) or by hyaluronic acid (HA-NPs).

FIG. 4 depicts the release of siRNA from HA_{DPA-Zn}-NPs after the addition of the salts indicated.

FIG. 5 depicts the cell viability of 4T1-fluc cells on treatment with HA-NPs and with HA_{DPA-Zn}-NPs.

FIG. 6 depicts in vitro enzyme-triggered release of DiO (3,3′-dioctadecyloxacarbocyanine perchlorate) from DiO/HA_{DPA-Zn}-NPs with 4T1-fluc and 293T cells.

FIG. 7 depicts in vivo optical images of fluc-expressing tumor after intratumor injection of siLuc/HA_{DPA-Zn}-NPs, HA_{DPA-Zn}-NPs, naked siLuc, or PBS.

FIG. 8 depicts quantitative analysis of fluc-expressing tumor after intratumor injection of siLuc/HA_{DPA-Zn}-NPs, HA_{DPA-Zn}-NPs, naked siLuc, or PBS.

FIG. 9 depicts the results of an electrophoretic retardation analysis of siRNA (a, b), miRNA (c) and oligonucleotide (d) (all at 10 pmol) binding with HA_{DPA-Zn} and CaP-HA_{DPA-Zn}-NP (all at 2 μg of HA_{DPA-Zn}-NP).

FIG. 10 depicts cellular images of HCT116 cells treated with Cy3-siRNA complexed with CaP-HA_{DPA-Zn}-NP (a) or Lipofectamine 2000 (Lipo2K) (b); CD44-blocked cells treated with CaP-HA_{DPA-Zn}/siRNA (c) or Cy3-siRNA only (d). Blue (DAPI staining, nuclei) and red (siRNA).

FIG. 11 depicts Quantitative FACS results. FIG. 11A provides the legend and intensity data and, FIG. 11B provides the staining pattern and fluorescence curves, FIG. 11C provides fluorescence images of DU145 cancer cells treated with CaP-HA_{DPA-Zn}/siRNA and Lipo2K/siRNA.

FIG. 12 depicts the suppression of fLuc gene expression and viability of 143B-fLuc cells after treatment with siRNA (siLuc or siNC) only, CaP-HA_{DPA-Zn}-NP or Lipo2K complexed with siLuc (FIG. 12A, FIG. C, and FIG. E) or siNC (silicon nanocrystal) (FIG. 12B, FIG. D, FIG. F). *p<0.005, **p<0.05 versus control or Lipo2K/siLuc.

FIG. 13 depicts the suppression of GFP gene expression of DU145-GFP cells by CaP-HA_{DPA-Zn}-NP or Lipo2K complexed with siGFP. *p<0.005 versus Control group or Lipo2K/siGFP group.

FIG. 14 depicts the suppression of fLuc signals in HCT116-fLuc-miR-34a cells by CaP-HA_{DPA-Zn}-NP (FIG. 14A and FIG. 14C) or LipoMAX (FIG. 14B, FIG. 14C) complexed with miR-34a, miR-NC or empty carriers without complexation. FIG. 14D depicts the viability of HCT116 cells after treatment of CaP-HA_{DPA-Zn} or LipoMAX complexed with miRNA (5 pmol) or empty carriers. *p<0.005, **p<0.05 versus the Control or LipoMAX/miRNA groups.

FIG. 15A depicts the multi-channel fluorescence images of DU145 cells treated with Cy5.5-labeled CaP-HA_{DPA-Zn}-NP complexed with Cy3-siRNA and loaded with Oregon green-conjugated paclitaxel (OG-PTX). FIG. 15B depicts the merged images depicted in FIG. 15A. FIG. 15C and FIG. 15D depict cellular images and co-localization efficiency of PTX/siRNA, siRNA/CaP-HA_{DPA-Zn} or PTX/CaP-HA_{DPA-Zn}.

### DETAILED DESCRIPTION OF THE INVENTION

The invention provides nanoparticulate complex comprising an artificial phosphate receptor of formula (I): P-[L-[-N(CH₂-2-pyridyl)₂]]ₚ.pZn²⁺ (I), wherein P represents a nanoparticulate substrate, L represents a linking group, and p is an integer of ≧1, in combination with an anion or anions.

In certain embodiments, the nanoparticulate substrate is an organic polymeric substrate, a biopolymeric substrate, or an inorganic substrate.

In certain embodiments, the nanoparticulate substrate comprises a biopolymeric substrate. In certain embodiments, the biopolymeric substrate comprises a polysaccharide.

Examples of suitable organic polymers include polyvinyl alcohol, polyoxyethylene diesters, and poly(meth)acrylates. Examples of suitable synthetic organic polymeric and biopolymeric substrates include poly(α-amino acids), albumin, other biopolymers, and polysaccharides.

Examples of suitable inorganic nanoparticles comprise core material formulations such as gold, silica, semiconductors, and metal oxides. Among them, superparamagnetic iron oxide NPs possess physicochemical and biological properties useful for drug delivery. Suitable inorganic nanoparticles are described in, e.g., Veiseh O. et al., Biomaterials 2011 August; 32(24):5717-5725 and references cited therein.

In a preferred embodiment, the nanoparticulate substrate comprises hyaluronic acid. Hyaluronic acid is a substrate for CD44, which is overexpressed on the surfaces of a variety of tumor cells. Thus, hyaluronic acid nanoparticles (HA-NPs) can actively accumulate in tumor cells. Once HA-NPs accumulate in tumor cells by passive and active targeting mechanisms, drugs encapsulated therein are released efficiently into the tumor cells through enzyme-triggered degradation of the nanoparticles by the hyaluronic acid-degrading intracellular enzyme hyaluronidase-1.

Hyaluronic acid can be used per se, or the hyaluronic acid can be functionalized using any suitable group. Examples of suitable groups include cholestanic acid and other steroidal molecules.

In certain preferred embodiments, the nanoparticulate substrate comprises the structure:

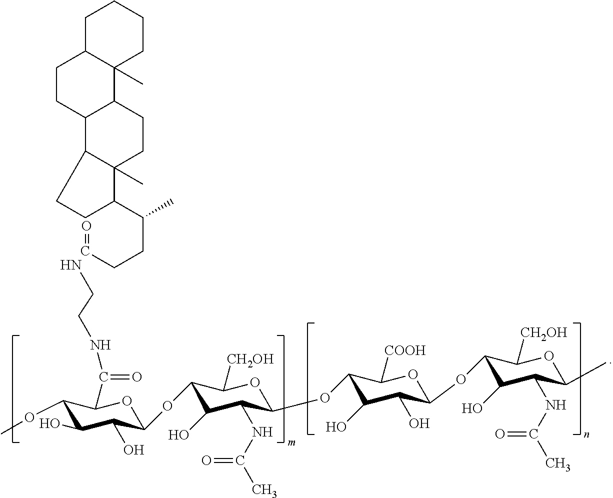

In any of the embodiments, L comprises a substituted or unsubstituted aryl group.

In certain embodiments, L comprises an Ω-(3,5-disubstituted aryl)alkylamino group.

In a certain preferred embodiment, L-[-N(CH₂-2-pyridyl)₂] is:

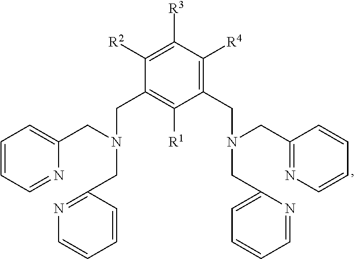

wherein R¹ is hydrogen or —OH, wherein R² and R⁴ are independently hydrogen or C₁-C₆ alkyl, and wherein R³ is straight or branched —NH-alkyl.

In a further preferred embodiments, L-[-N(CH₂-2-pyridyl)ₚ] is:

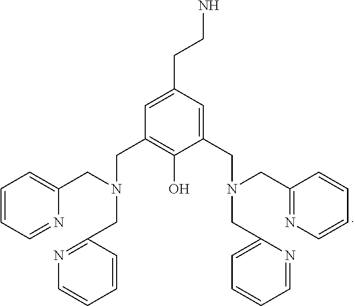 [SMILES: NCCc1cc(CN(Cc2ccccn2)Cc2ccccn2)c(O)c(CN(Cc2ccccn2)Cc2ccccn2)c1]

In any of the above embodiments, the artificial phosphate receptor has the formula:

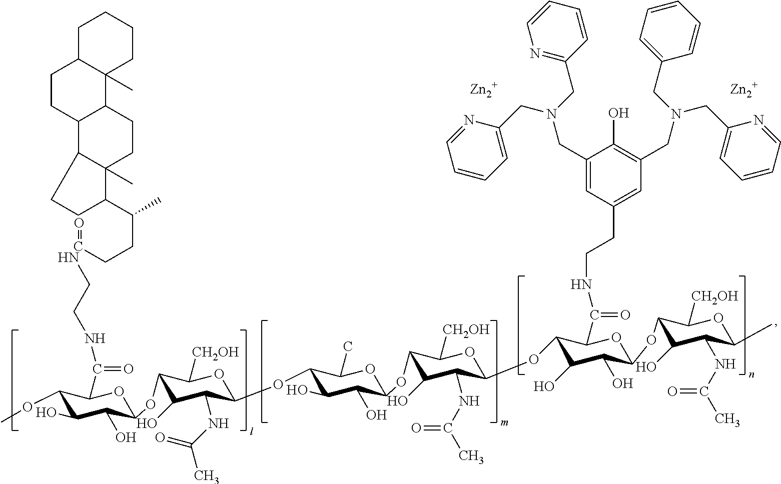

wherein l, m, and n are independently integers of from 1 to about 10,000.

In certain preferred embodiments, the ratio of n to (1+m) is from 0.01 to about 1.0. In certain more preferred embodiments, the ratio of n to (1+m) is from 0.1 to about 0.5.

p can be any suitable integer, 1 or greater than 1. For example, p is an integer of 1 to about 500,000, e.g., 1 to about 250,000, or 1 to about 100,000, or 1 to about 50,000, or 1 to about 25,000, or 1 to about 10,000, or 1 to about 5,000, or 1 to about 1,000, or 1 to about 500, or 1 to about 100.

Referring now to terminology used generically herein, the term “alkyl” means a straight-chain or branched alkyl group containing from, for example, 1 to about 6 carbon atoms, preferably from 1 to about 4 carbon atoms, more preferably from 1 to 2 carbon atoms. Examples of such substituents include methyl, ethyl, propyl, isopropyl, n-butyl, sec-butyl, isobutyl, tert-butyl, pentyl, isoamyl, hexyl, and the like.

The term “aryl” refers to an unsubstituted or substituted aromatic carbocyclic substituent, as commonly understood in the art, and includes phenyl. It is understood that the term aryl applies to cyclic substituents that are planar and comprise 4n+2π electrons, according to Hückel's Rule.

The invention provides a phosphate anion ligand complex comprising at least one phosphate anion ligand complexed with the nanoparticulate complex as described herein. In certain embodiments, the phosphate anion ligand is selected from siRNA (small interfering RNA), miRNA (micro RNA), oligonucleotides, RNA, and DNA. In preferred embodiments, the phosphate anion ligand is siRNA.

All of the suitable phosphate anion ligands contain anionic phosphate moieties. It is believed that specific interactions between the coordinated zinc ions of the dipicolylamine group and the anionic phosphate moieties allows for selective binding of the phosphate anion ligands with the nanoparticulate complex described herein.

Hyaluronic acid nanoparticles that are conjugated with zinc-dipicolylamine moieties (referred to herein as HA_{DPA-Zn}-NPs) have a high affinity for phosphate ligands such as siRNA. This affinity improves the targeting of the system for gene silencing and maintains the advantages of HA-NPs for cellular delivery of the siRNA, miRNA, or other oligonucleotides. Polymeric nanoparticles functionalized with Zn-dipicolylamine (Zn-DPA or DPA-Zn) provide highly targeted small-molecule delivery and efficient intracellular transfer of siRNA, miRNA, or other oligonucleotides, with low toxicity.

The invention further provides an anticancer complex comprising an anticancer agent and the phosphate anion ligand complex described herein. The anticancer agent can be loaded onto the surface and/or the inner core of the nanoparticles using any suitable method for incorporating the anticancer agent onto or within the nanoparticles. In addition, as siRNA and hydrophobic anticancer drugs can be simultaneously loaded onto the surface and the inner core of HA_{DPA-Zn}-NPs, this system can be used as a co-delivery carrier to maximize and synergize therapeutic effects.

Chemistry

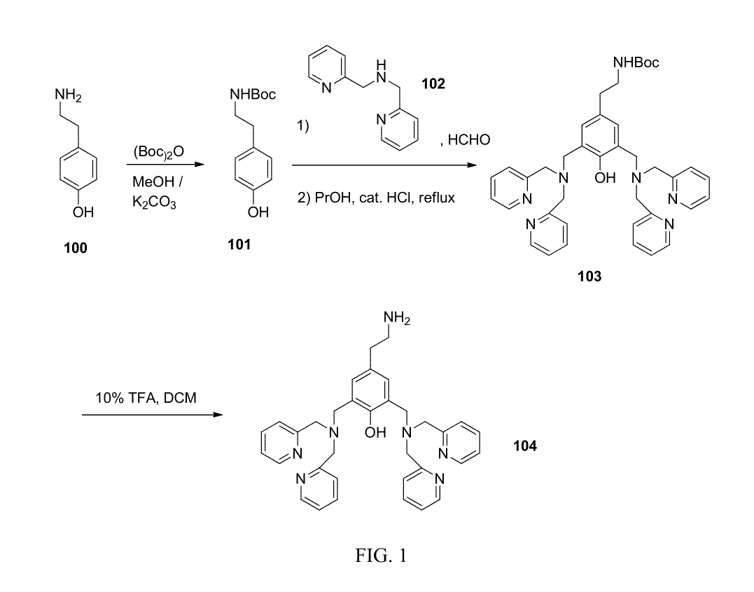

Dipicolylamine derivative 104 can be prepared as depicted in FIG. 1: (a) protection of the amino group of (4-hydroxyphenyl-ethylamine 100 with, e.g., (Boc)₂O in a solvent such as methanol in the presence of a base such as potassium carbonate provides protected amine 101; (b) reaction of the compound 101 with dipicolylamine 102 and formaldehyde gives compound 103; and (c) deprotection of compound 103 by treatment with for example, 10% trifluoroacetic acid in dichloromethane gives compound 104.

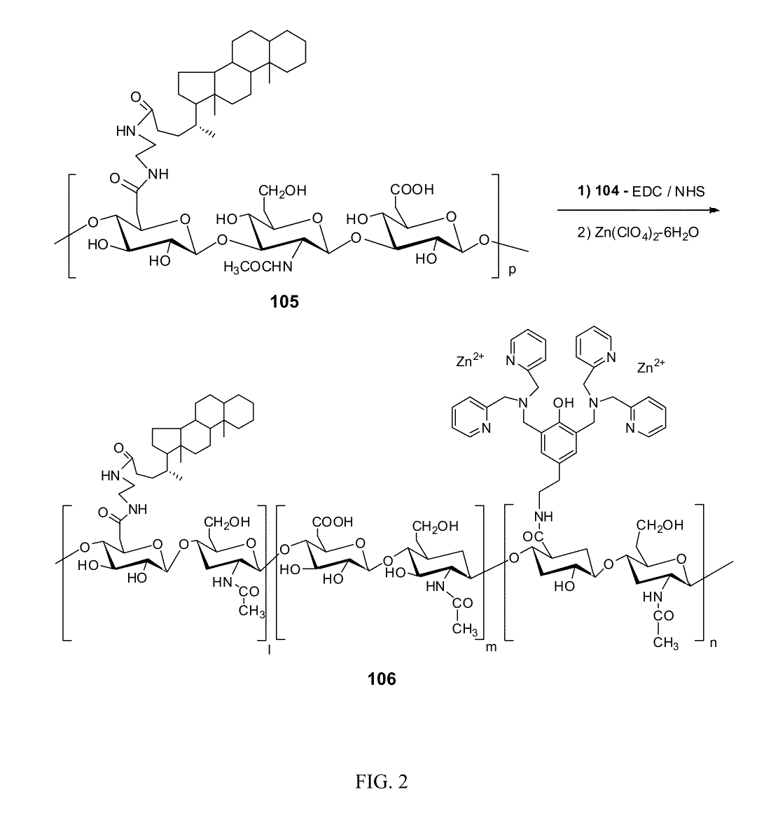

The artificial phosphate receptor of formula (I) can be prepared as depicted in FIG. 2. Covalent conjugation of the bisdipicolylamine derivative 104 to the carboxyl groups of cholestanic acid-functionalized HA-NP 105 using, e.g., ethyl(dimethylaminopropyl)carbodiimide/N-hydroxysuccinamide activation in a buffer solution provides a HA_{DPA-Zn}-NP conjugate. After dialysis against distilled water, zinc ions can be incorporated into the complex to give 106 by the addition of, e.g., excess Zn(ClO₄)₂-6H₂O and sonication. The conjugate 106 can be isolated as a white powder by lyophilization. HA-NP 105 can be prepared as described in Choi K. Y. et al., Biomaterials 2010, 31, 106-114.

The invention also provides an anticancer complex comprising an anticancer agent and the phosphate anion ligand complex described herein. The anticancer agent can be chosen from reversible DNA binders, DNA alkylators, antineoplastic alkylating agents, and DNA strand breakers. Examples of suitable reversible DNA binders include topetecan hydrochloride, irinotecan (CPT11—Camptosar), rubitecan, exatecan, nalidixic acid, TAS-103, etoposide, acridines (e.g., amsacrine, aminocrine), actinomycins (e.g., actinomycin D), anthracyclines (e.g., doxorubicin, daunorubicin), benzophenainse, XR 11576/MLN 576, benzopyridoindoles, Mitoxantrone, AQ4, Etopside, Teniposide, (epipodophyllotoxins), and bisintercalating agents such as triostin A and echinomycin.

Examples of suitable DNA alkylators include sulfur mustard, the nitrogen mustards (e.g., mechlorethamine), chlorambucil, melphalan, ethyleneimines (e.g., triethylenemelamine, carboquone, diaziquone), methyl methanesulfonate, busulfan, CC-1065, duocarmycins (e.g., duocarmycin A, duocarmycin SA), triazine antitumor drugs such as triazenoimidazole (e.g., dacarbazine), mitomycin C, leinamycin, and the like.

Examples of suitable DNA strand breakers include doxorubicin and daunorubicin (which are also reversible DNA binders), other anthracyclines, belomycins, tirapazamine, enediyne antitumor antibiotics such as neocarzinostatin, esperamicins, calicheamicins, dynemicin A, hedarcidin, C-1027, N1999A2, esperamicins, zinostatin, and the like.

Antineoplastic alkylating agents are agents that alkylate the O⁶-position of guanine residues in DNA. Examples of antineoplastic alkylating agents include chloroethylating agents. The most frequently used chloroethylating agents include 1-(2-chloroethyl)-3-cyclohexyl-1-nitrosourea (CCNU, lomustine), 1,3-bis(2-chloroethyl)-1-nitrosourea (BCNU, carmustine), 1-(2-chloroethyl)-3-(4-methylcyclohexyl)-1-nitrosourea (MeCCNU, semustine), and 1-(2-chloroethyl)-3-(4-amino-2-methyl-5-pyrimidinyl)methyl-1-nitrosourea (ACNU). Such agents have been used clinically against tumors of the central nervous system, multiple myeloma, melanoma, lymphoma, gastrointestinal tumors, and other solid tumors (Colvin and Chabner, Alkylating Agents. In: Cancer Chemotherapy: Principles and Practice. Edited by B. A. Chabner and J. M. Collins, Lippincott, Philadelphia, Pa. pp. 276-313 (1990); and McCormick et al., Eur. J Cancer 26: 207-221 (1990)).

Chloroethylating agents, which have fewer side effects and are currently under development include 1-(2-chloroethyl)-3-(2-hydroxyethyl)-1-nitrosourea (HECNU), 2-chloroethylmethylsulfonylmethanesulfonate (Clomesone), and 1-[N-(2-chloroethyl)-N-nitrosoureido]ethylphosphonic acid diethyl ester (Fotemustine) (Colvin and Chabner (1990), supra; and McCormick et al. (1990), supra). Methylating agents include Streptozotocin (2-deoxy-2-(3-methyl-3-nitrosoureido)-D-glucopyranose), Procarbazine (N-(1-methylethyl)-4-[(2-methylhydrazino)methyl]benzamide), Dacarbazine or DTIC (5-(3,3-dimethyl-1-triazenyl)-1H-imidazole-4-carboxamide), and Temozolomide (8-carbamoyl-3-methylimidazo[5.1-d]-1,2,3,5-tetrazin-4-(3H)-one).

Temozolomide is active against malignant melanomas, brain tumors and mycosis fungoides. Streptozotocin is effective against pancreatic tumors. Procarbazine is used to treat Hodgkin's disease and brain tumors. DTIC is used to treat melanoma and lymphomas (Colvin and Chabner (1990), supra; and Longo, Semin. Concol. 17: 716-735 (1990)).

Other non-limiting examples of suitable anticancer agents include abarelix, aldesleukin, alemtuzumab, altretamine, amifostine, aminoglutethimide, anastrazole, arsenic trioxide, asparaginase, azacitidine, azathioprine, BCG vaccine, bevacizumab, bexarotene, bicalutamide, bleomycin sulfate, bortezomib, bromocriptine, busulfan, capecitabine, carboplatin, carmustine, cetuximab, chlorambucil, chloroquine phosphate, cladribine, cyclophosphamide, cyclosporine, cytarabine, dacarbazine, dactinomycin, daunorubicin hydrochloride, daunorubicin citrate liposomal, dexrazoxane, docetaxel, doxorubicin hydrochloride, doxorubicin hydrochloride liposomal, epirubicin hydrochloride, estramustine phosphate sodium, etoposide, estretinate, exemestane, floxuridine, fludarabine phosphate, fluorouracil, fluoxymesterone, flutamide, fulvestrant, gefitinib, gemcitabine hydrochloride, gemtuzumab ozogamicin, goserelin acetate, hydroxyurea, idarubicin hydrochloride, ifosfamide, imtinib mesylate, interferon alfa-2a, interferon alfa-2b, irinotecan hydrochloride trihydrate, letrozole, leucovorin calcium, leuprolide acetate, levamisole hydrochloride, lomustine, lymphocyte immune anti-thymocyte globulin (equine), mechlorethamine hydrochloride, medoxyprogestone acetate, melphalan, mercaptopurine, mesna, methotrexate, mitomycin, mitotane, mitoxantrone hydrochloride, nilutamide, oxaliplatin, paclitaxel, pegaspargase, pentostatin, plicamycin, porfimer sodium, procarbazine hydrochloride, streptozocin, tamoxifen citrate, temozolomide, teniposide, testolactone, testosterone propionate, thioguaine, thiotepa, topotecan hydrochloride, tretinoin, uracil mustard, valrubicin, vinblastine sulfate, vincristine sulfate, and vinorelbine.

The present invention further provides a pharmaceutical composition comprising a pharmaceutically acceptable carrier and at least one DPA/Zn-functionalized nanoparticulate complex, phosphate anion ligand complex comprising at least one phosphate anion ligand complexed with a DPA/Zn-functionalized nanoparticulate complex, or anticancer complex comprising an anticancer agent and the phosphate anion ligand complexed with a DPA/Zn-functionalized nanoparticulate complex.

In certain embodiments, the pharmaceutical composition may comprise a pharmaceutically acceptable carrier and at least one DPA/Zn-functionalized nanoparticulate complex or phosphate anion ligand complex comprising at least one phosphate anion ligand complexed with a DPA/Zn-functionalized nanoparticulate complex and an anticancer agent which is co-administered separately from the DPA/Zn-functionalized nanoparticulate complex. In accordance with some embodiments, the DPA/Zn-functionalized nanoparticulate complex or phosphate anion ligand complex comprising at least one phosphate anion ligand complexed with a DPA/Zn-functionalized nanoparticulate complex is administered in combination with an anticancer agent or combination of anticancer agents. For example, in some embodiments, the combinatorial formulation may include one or more anticancer agents as described herein in combination with a compound of formula (I), among other combinations. In other embodiments, the combinatorial formulation may include one or more additional chemotherapeutic agents.

To practice coordinate administration methods of the invention, a DPA/Zn-functionalized nanoparticulate complex or phosphate anion ligand complex comprising at least one phosphate anion ligand complexed with a DPA/Zn-functionalized nanoparticulate complex may be administered, simultaneously or sequentially, or cyclically, in a coordinate treatment protocol with one or more of the anticancer agents contemplated herein. Thus, in certain embodiments a DPA/Zn-functionalized nanoparticulate complex or phosphate anion ligand complex comprising at least one phosphate anion ligand complexed with a DPA/Zn-functionalized nanoparticulate complex is administered coordinately with a different agent, or any other secondary or adjunctive therapeutic agent contemplated herein, using separate formulations or a combinatorial formulation as described above (i.e., comprising both a DPA/Zn-functionalized nanoparticulate complex or phosphate anion ligand complex comprising at least one phosphate anion ligand complexed with a DPA/Zn-functionalized nanoparticulate complex or related or derivative compound, and another anticancer agent). This coordinated administration may be done simultaneously or sequentially in either order, and there may be a time period while only one or both (or all) active therapeutic agents individually and/or collectively exert their biological activities.

It is preferred that the pharmaceutically acceptable carrier be one that is chemically inert to the active complex and one that has no detrimental side effects or toxicity under the conditions of use.

The choice of carrier will be determined in part by the particular complex of the present invention chosen, as well as by the particular method used to administer the complex. Accordingly, there is a wide variety of suitable formulations of the pharmaceutical composition of the present invention. The following formulations for oral, aerosol, nasal, pulmonary, parenteral, subcutaneous, intravenous, intramuscular, intraperitoneal, intrathecal, intratumoral, topical, rectal, and vaginal administration are merely exemplary and are in no way limiting.

The pharmaceutical composition can be administered parenterally, e.g., intravenously, subcutaneously, intradermally, or intramuscularly. Thus, the invention provides compositions for parenteral administration that comprise a solution or suspension of the inventive complex dissolved or suspended in an acceptable carrier suitable for parenteral administration, including aqueous and non-aqueous isotonic sterile injection solutions.

Overall, the requirements for effective pharmaceutical carriers for parenteral compositions are well known to those of ordinary skill in the art. See, e.g., Banker and Chalmers, eds., Pharmaceutics and Pharmacy Practice, J. B. Lippincott Company, Philadelphia, pp. 238-250 (1982), and Toissel, ASHP Handbook on Injectable Drugs, 4th ed., pp. 622-630 (1986). Such solutions can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The complex of the present invention may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) DPA/Zn detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

The parenteral formulations can contain preservatives and buffers. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Topical formulations, including those that are useful for transdermal drug release, are well-known to those of skill in the art and are suitable in the context of the invention for application to skin. Topically applied compositions are generally in the form of liquids, creams, pastes, lotions and gels. Topical administration includes application to the oral mucosa, which includes the oral cavity, oral epithelium, palate, gingival, and the nasal mucosa. In some embodiments, the composition contains at least one active component and a suitable vehicle or carrier. It may also contain other components, such as an anti-irritant. The carrier can be a liquid, solid or semi-solid. In embodiments, the composition is an aqueous solution. Alternatively, the composition can be a dispersion, emulsion, gel, lotion or cream vehicle for the various components. In one embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In embodiments of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

Formulations suitable for oral administration can consist of (a) liquid solutions, such as a therapeutically effective amount of the inventive compound dissolved in diluents, such as water, saline, or orange juice, (b) capsules, sachets, tablets, lozenges, and troches, each containing a predetermined amount of the active ingredient, as solids or granules, (c) powders, (d) suspensions in an appropriate liquid, and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The nanoparticulate complex of the present invention, alone or in combination with other suitable components, can be made into aerosol formulations to be administered via inhalation. The nanoparticulate complexes are preferably supplied in finely divided form along with a surfactant and propellant. Typical percentages of active complex are 0.01%-20% by weight, preferably 1%-10%. The surfactant must, of course, be nontoxic, and preferably soluble in the propellant. Representative of such surfactants are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25%-5%. The balance of the composition is ordinarily propellant. A carrier can also be included as desired, e.g., lecithin for intranasal delivery. These aerosol formulations can be placed into acceptable pressurized propellants, such as dichlorodifluoromethane, propane, nitrogen, and the like. They also may be formulated as pharmaceuticals for non-pressured preparations, such as in a nebulizer or an atomizer. Such spray formulations may be used to spray mucosa.

Additionally, the complex of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

It will be appreciated by one of ordinary skill in the art that, in addition to the aforedescribed pharmaceutical compositions, the complex of the present invention may be formulated as inclusion complexes, such as cyclodextrin inclusion complexes, or liposomes. Liposomes serve to target the complexes to a particular tissue, such as lymphoid tissue or cancerous hepatic cells. Liposomes can also be used to increase the half-life of the inventive complex. Liposomes useful in the present invention include emulsions, foams, micelles, insoluble monolayers, liquid crystals, phospholipid dispersions, lamellar layers and the like. In these preparations, the active complex to be delivered is incorporated as part of a liposome, alone or in conjunction with a suitable chemotherapeutic agent. Thus, liposomes filled with a desired inventive complex, can be directed to the site of a specific tissue type, hepatic cells, for example, where the liposomes then deliver the selected compositions. Liposomes for use in the invention are formed from standard vesicle-forming lipids, which generally include neutral and negatively charged phospholipids and a sterol, such as cholesterol. The selection of lipids is generally guided by consideration of, for example, liposome size and stability of the liposomes in the blood stream. A variety of methods are available for preparing liposomes, as described in, for example, Szoka et al., Ann. Rev. Biophys. Bioeng., 9, 467 (1980), and U.S. Pat. Nos. 4,235,871, 4,501,728, 4,837,028, and 5,019,369. For targeting to the cells of a particular tissue type, a ligand to be incorporated into the liposome can include, for example, antibodies or fragments thereof specific for cell surface determinants of the targeted tissue type. A liposome suspension containing a compound or salt of the present invention may be administered intravenously, locally, topically, etc. in a dose that varies according to the mode of administration, the agent being delivered, and the stage of disease being treated.

In certain embodiments, the invention provides a method of silencing a gene in a cancer patient in need thereof comprising administering an effective amount of the nanoparticulate complex, the anticancer complex, or the pharmaceutical composition described herein. In certain embodiments, the invention provides a method for treating or preventing cancer in a patient in need thereof, comprising administering an effective amount of the complex, the anticancer complex, or the pharmaceutical composition described herein. In certain embodiments, the invention provides a method for targeting a cell in cancer treatment, comprising administering an effective amount of the complex, the anticancer complex, or the pharmaceutical composition described herein.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the inventive methods can provide any amount of any level of treatment, prevention, amelioration, or inhibition of the disorder in a mammal. For example, a disorder, including symptoms or conditions thereof, may be reduced by, for example, 100%, 90%, 80%, 70%, 60%, 50%, 40%, 30%, 20%, or 10%. Furthermore, the treatment, prevention, amelioration, or inhibition provided by the inventive method can include treatment, prevention, amelioration, or inhibition of one or more conditions or symptoms of the disorder, e.g., cancer. Also, for purposes herein, “treatment,” “prevention,” “amelioration,” or “inhibition” can encompass delaying the onset of the disorder, or a symptom or condition thereof.

In accordance with the invention, the term “animal” includes a mammal such as, without limitation, the order Rodentia, such as mice, and the order Lagomorpha, such as rabbits. It is preferred that the mammals are from the order Carnivora, including Felines (cats) and Canines (dogs). It is more preferred that the mammals are from the order Artiodactyla, including Bovines (cows) and Swine (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The nanoparticulate complex, phosphate anion ligand complex, and/or anticancer complex is administered in a dose sufficient to treat the cancer. Such doses are known in the art (see, for example, the Physicians' Desk Reference (2004)). The nanoparticulate complex, phosphate anion ligand complex, and/or anticancer complex can be administered using techniques such as those described in, for example, Wasserman et al., Cancer, 36, pp. 1258-1268 (1975) and Physicians' Desk Reference, 58th ed., Thomson PDR (2004).

Suitable doses and dosage regimens can be determined by conventional range-finding techniques known to those of ordinary skill in the art. Generally, treatment is initiated with smaller dosages that are less than the optimum dose of the nanoparticulate complex, phosphate anion ligand complex, and/or anticancer complex of the present invention. Thereafter, the dosage is increased by small increments until the optimum effect under the circumstances is reached. The present method can involve the administration of about 0.1 μg to about 50 mg of at least one nanoparticulate complex, phosphate anion ligand complex, and/or anticancer complex of the invention per kg body weight of the individual. For a 70 kg patient, dosages of from about 10 μg to about 200 mg of the nanoparticulate complex, phosphate anion ligand complex, and/or anticancer complex of the invention would be more commonly used, depending on a patient's physiological response.

By way of example and not intending to limit the invention, the dose of the nanoparticulate complex, phosphate anion ligand complex, and/or anticancer complex described herein for methods of treating or preventing a disease or condition as described above can be about 0.001 to about 1 mg/kg body weight of the subject per day, for example, about 0.001 mg, 0.002 mg, 0.005 mg, 0.010 mg, 0.015 mg, 0.020 mg, 0.025 mg, 0.050 mg, 0.075 mg, 0.1 mg, 0.15 mg, 0.2 mg, 0.25 mg, 0.5 mg, 0.75 mg, or 1 mg/kg body weight per day. The dose of the nanoparticulate complex described herein for the described methods can be about 1 to about 1000 mg/kg body weight of the subject being treated per day, for example, about 1 mg, 2 mg, 5 mg, 10 mg, 15 mg, 0.020 mg, 25 mg, 50 mg, 75 mg, 100 mg, 150 mg, 200 mg, 250 mg, 500 mg, 750 mg, or 1000 mg/kg body weight per day.

In certain embodiments, the invention provides a nanoparticulate complex comprising an artificial phosphate receptor of formula (I): P-[L-[-N(CH₂-2-pyridyl)₂]]ₚ.pZn²⁺ (I) wherein P represents a nanoparticulate substrate, L represents a linking group, and p is an integer of ≧1, for use in treating cancer.

In certain embodiments, the invention provides a kit comprising a nanoparticulate complex comprising an artificial phosphate receptor of formula (I): P-[L-[-N(CH₂-2-pyridyl)₂]]ₚ.pZn²⁺ (I), wherein P represents a nanoparticulate substrate, L represents a linking group, and p is an integer of ≧1, at least one nucleic acid, and optionally at least one anticancer agent, and instructions for use thereof.

In these embodiments, an article of manufacture, or “kit”, containing materials useful for the treatment of the diseases and disorders described above is provided. In one embodiment, the kit comprises a container comprising a DPA/Zn-functionalized nanoparticulate complex, a container comprising at least one anticancer agent, or a container comprising a composition comprising a complex and at least one anticancer agent. The kit may further comprise a label or package insert, on or associated with the container(s). The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, and the like. The container(s) may be formed from a variety of materials such as glass or plastic. The container(s) may have a sterile access port (for example, the container may be an intravenous solution bag or a vial having a stopper pierceable by a hypodermic injection needle). At least one active agent in the composition is a nanoparticular complex of formula (I). The label or package insert indicates that the composition is used for treating the condition of choice, such as cancer. The label or package insert may also indicate that the composition can be used to treat other disorders. Alternatively, or additionally, the article of manufacture may further comprise a second or third container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain other embodiments wherein the kit comprises a DPA/Zn-functionalized nanoparticulate complex and a separate composition comprising an anticancer agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

The following examples further illustrate the invention but, of course, should not be construed as in any way limiting its scope.

### Example 1

This example demonstrates a method for synthesis of a DPA/Zn-functionalized nanoparticulate complex in accordance with an embodiment of the invention.

Synthesis of HA_{DPA-Zn}-NP: Bis(DPA) analog 104 was chemically conjugated to HA-NP 105 (m.w. 234,000 Da). The amphiphilic HA conjugate bearing ten 5β-cholanic acids per 100 sugar residues of HA was made through amide formation in the presence of EDC and NHS which was mixed with EDC (3 mg, 15.6 mmol) in PBS and HOBt (3 mg, 22.2 μmol) in methanol. After 104 (5 mg, 9 μmol) was slowly added, the mixture was stirred for 24 h at room temperature. The resulting solution was dialyzed against distilled water for 24 h. After being freeze-dried, the HA_{DPA}-NP conjugate was isolated as a white powder. HA_{DPA-Zn}-NP 106 was prepared by mixing HA_{DPA}-NP with an appropriate amount of Zn ions in HEPES buffer under agitation and the resulting mixture was incubated at room temperature for 20 min. The structure and purity of 104 were analyzed by ¹H/¹³C NMR and RP-HPLC. 106 was analyzed by ¹H NMR and the loading of 104 on the HA-NP was calculated. The samples were prepared by dissolving 106 in D₂O/CD₃OD (1 v/1 v). The characteristic peaks of HA were primarily found at 2.0 ppm (the methyl group at the C2 position of N-acetyl glucosamine) and 3.3-4.8 ppm (methylene and hydroxyl groups at the sugar unit), whereas those of CA appeared in the range of 0.6-1.8 ppm (methyl and methylene groups of the ring structure). Successful conjugation of 104 was confirmed by the peak appearing at 6.8-8.3 ppm (the aromatic CH). The amount of 104 in 105 was quantitatively characterized by the integration ratio between the characteristic peaks of HA at 2.0 ppm and 104 at 6.8-8.3 ppm. 32 molecules of 104 were conjugated to 100 repeating units of HA, and the molecular weight of the resulting 106 was 274,000 Da. Nanoparticle formations of 105 and 106 were analyzed by TEM. Size distributions of each formulation were measured by dynamic light scattering (DLS).

¹H NMR spectrum of 104 (300 MHz, CDCl3): δ=8.52 (d, J (H, H)=4.5 Hz, 4H), 7.60 (t, J (H, H)=7.5 Hz, 4H), 7.50 (d, J (H, H)=7.7 Hz, 4H), 7.12 (t, J (H, H)=6.0 Hz, 4H), 7.03 (s, 2H), 3.87 (s, 8H), 3.79 (s, 4H), 2.89 (t, J (H, H)=6.7 Hz, 2H), 2.63 ppm (t, J (H, H)=6.7 Hz, 2H).

¹³C NMR spectrum of 104 (300 MHz, CDCl3): δ=159.2, 154.3, 148.9, 136.5, 129.5, 124.0, 122.9, 122.3, 122.0, 59.8, 54.8, 43.6, 39.0.

### Example 2

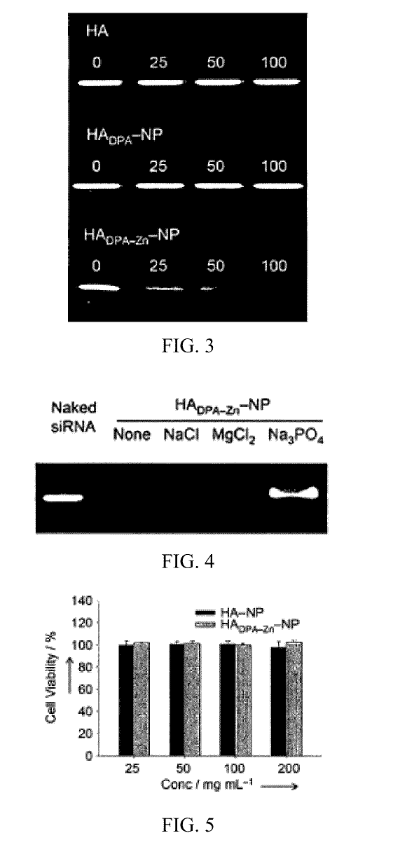

The ability of HA_{DPA-Zn}-NPs to form Zn-DPA (dipicolylamine)-mediated complexes with siRNA was investigated by using a siRNA (siLuc) that targets the firefly luciferase (flue) gene as a model compound. Different amounts of HA and HPs with zinc (HA_{DPA-Zn}-NPs) and without zinc (HA_{DPA}-NPs) were mixed with 15 pmol of siLuc and analyzed in a retardation assay by agarose gel electrophoresis as described below. As shown in FIG. 3, the HA_{DPA-Zn}-NPs were able to bind siLuc but HA and the HA_{DPA}-NPs alone did not bind to siLuc at any concentration. To confirm that this complex structure was based on coordination between the phosphate groups of the siRNA and Zn-DPA, excess sodium phosphate was added to induce decomplexation of the siLuc from the HA_{DPA-Zn}-NPs (FIG. 4). After the addition of sodium phosphate, siLuc was released from HA_{DPA-Zn}-NPs, whereas no significant change was observed after the addition of other salts. This result indicates that siLuc was bound on the HA_{DPA-Zn}-NPs by coordination with Zn-DPA.

The agarose gel electrophoresis was performed as follows: siRNA complexes were analyzed by 2% agarose gel electrophoresis. The gels were prepared with 2% agarose in Tris-acetate-EDTA buffer containing 0.5 μg/mL ethidium bromide and 2 mM of zinc ion. As for gel retardation assay, samples were incubated at room temperature for 20 min, after which an appropriate amount of DNA loading buffer was added to each sample. Gel electrophoresis was carried out at 100 V for 15 min and the gel was subsequently imaged using a LAS-3000 gel documentation system (Fujifilm Life Science, Japan). To assess the decomplexation of siRNA, HA_{DPA-Zn}-NP/siRNA was incubated with 0.1 M of sodium phosphates, magnesium chloride or sodium chloride for 15 min and the released siRNA fraction was imaged as described above.

### Example 3

This example demonstrates the cellular uptake of siRNA/HA_{DPA-Zn}-NP in cells that were strongly positive for CD44 (4T1-fluc) and cells that were less-strongly positive for CD44 (293T). CD44 expression was confirmed by flow cytometry. The red fluoresecence from Cy3-labeled siRNA was more intense in 4T1-fluc cells that were treated with siRNA/HA_{DPA-Zn}-NPs than in the cells that were treated with Lipofectamine 2000 (Lipo2K)/siRNA. In contrast, no fluorescence was observed in cells that were treated with HA, HA_{DPA}-NPs, and Zn-DPA. Furthermore, the uptake of siRNA/HA_{DPA-Zn}-NPs into 4T1-fluc cells was 3.7±0.5-fold higher than the uptake into 293T cells.

To determine whether CD44 receptors are responsible for the efficient cellular uptake of siRNA/HA_{DPA-Zn}-NPs, the CD44 receptors were blocked with excess HA or the anti-CD44 antibody HERM-1. Additionally, to investigate whether the cellular uptake of NPs is energy-dependent and proceeds by endocytosis, the active transport processes were strongly inhibited by using a low temperature or by introducing metabolic inhibitors. Each of the two cell lines were treated with siRNA/HA_{DPA-Zn}-NPs and incubated at 4° C. without a metabolic inhibitor or at 37° C. with the metabolic inhibitor sodium azide. All of the treatments clearly reduced the efficiency of siRNA/HA_{DPA-Zn}-NPs uptake into 4T1-fluc cells (36.0±6.2%, 28.7±1.5%, 28.3±3.5%, and 26.3±3.1%) for HA, HERM-1, 4° C., and sodium azide, respectively, which demonstrated that HA_{DPA-Zn}-NP/siRNA complexes enter the cells to some extent through CD44 receptor mediated endocytosis, and also in an energy-dependent manner.

### Example 4

The ability of HA_{DPA-Zn}-NPs to deliver siRNA was confirmed by treating 4T1-fluc cells with siLuc/HA_{DPA-Zn}-NPs and measuring the expression of fluc. The bioluminescence imaging (BLI) signal intensity of untreated 4T1-fluc cells was set to 100% flue expression.

In agreement with the uptake of Cy3-siRNA, siLuc/HA_{DPA-Zn}-NPs showed a remarkably high gene silencing efficacy in a dose-dependent manner (10 μg, 20 μg, and 40 μg of siLuc/HA_{DPA}Zn-NPs reduced the expression of fluc to (54.9±5.6)%, (33.4±3.5)%, and (10.5±1.2)%, respectively). The HA_{DPA-Zn}-NP/siLuc complex was (2.2±0.4)-fold more effective in silencing the expression of fluc than the Lipo2K formulation. To rule out the possibility that the decreased expression of fluc was caused by a reduction in cell viability because of nonspecific cytotoxicity, an MTT assay was performed to assess cell viability after the BLI experiment. No significant cytotoxicity was detected in the 4T1-fluc cells after BLI, which confirmed that the gene silencing effect was a consequence of the treatment with HA_{DPA-Zn}-NP/siLuc and was not caused by a reduced number of viable cells. The cell viability is depicted in FIG. 5.

### Example 5

This example demonstrates that HA_{DPA-Zn}-NPs can be used as a co-delivery carrier for both an anticancer drug and for siRNA. The hydrophobic and fluorescent carbocyanine 3,3′-dioctadecyloxacarbocyanine perchlorate (DiO) was used as a model compound. As DiO is a lipophilic molecule, it was readily encapsulated in HA_{DPA-Zn}-NPs in its fluorescently quenched state. The release of DiO from the NPs was monitored by measuring changes in fluorescence. The encapsulation of DiO along with siRNA in the HA_{DPA-Zn}-NPs slightly increased the mean diameter of the HA_{DPA-Zn}-NPs to (290.6±31.7) nm. After the siRNA/DiO/HA_{DPA-Zn}-NPs complex was exposed to a solution of buffer that contained Hyal-1 (120 units mL⁻¹), the fluorescence intensity increased significantly and was saturated within 30 min as a result of the release of DiO from the HA_{DPA-Zn}-NPs.

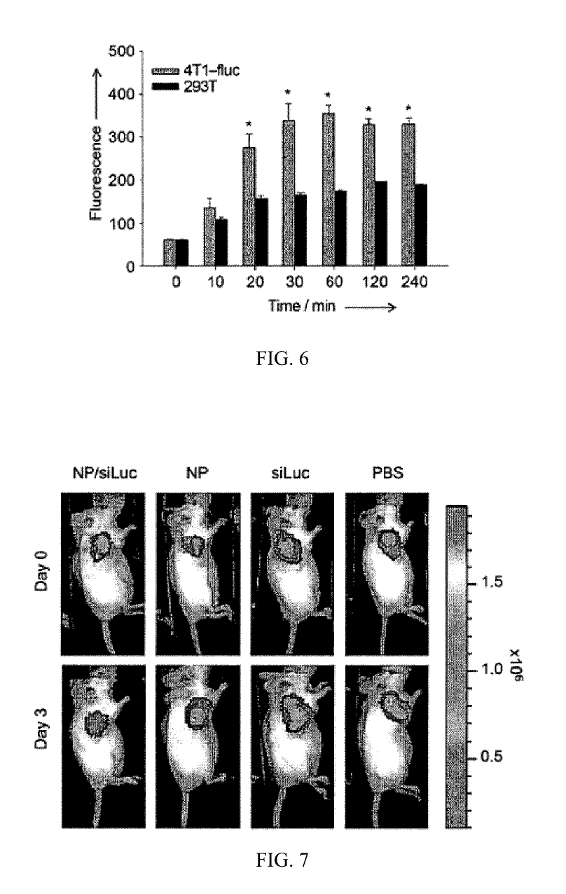

To monitor the release of DiO in cells, 4T1-fluc and 293T cells were treated with siRNA/DiO/HA_{DPA-Zn}-NPs, and the fluorescence intensity was recorded by a fluorescence microplate reader. As shown in FIG. 6, a gradual increase in the fluorescence occurred as early as 10 min after the cells were treated with siRNA/DiO/HA_{DPA-Zn}-NPs. The maximum fluorescence signal was detected after 30 min in 4T1-fluc cells. This implies that 4T1-fluc cells are highly permeable to the HA_{DPA-Zn}-NPs and that the HA_{DPA-Zn}-NPs are highly susceptible to intracellular Hyal-1, which readily destroys the structural integrity of the HA_{DPA-Zn}-NPs and leads to rapid release of the payload.

### Example 6

To verify if the intracellular release of DiO is Hyal-1-dependent, the release of DiO was also tested in NIH3T3 cells, which have a lower level of intracellular Hyal-1. The results demonstrate that the release of DiO was significantly lower in NIH3T3 cells relative to 4T1-fluc cells. The finding that HA_{DPA-Zn}-NPs enhance cellular uptake of both encapsulated drugs and siRNAs that are coordinated to Zn-DPA was supported by confocal microscopy images of 4T1-fluc cells after treatment with siRNA/DiO/HA_{DPA-Zn}-NPs.

### Example 7

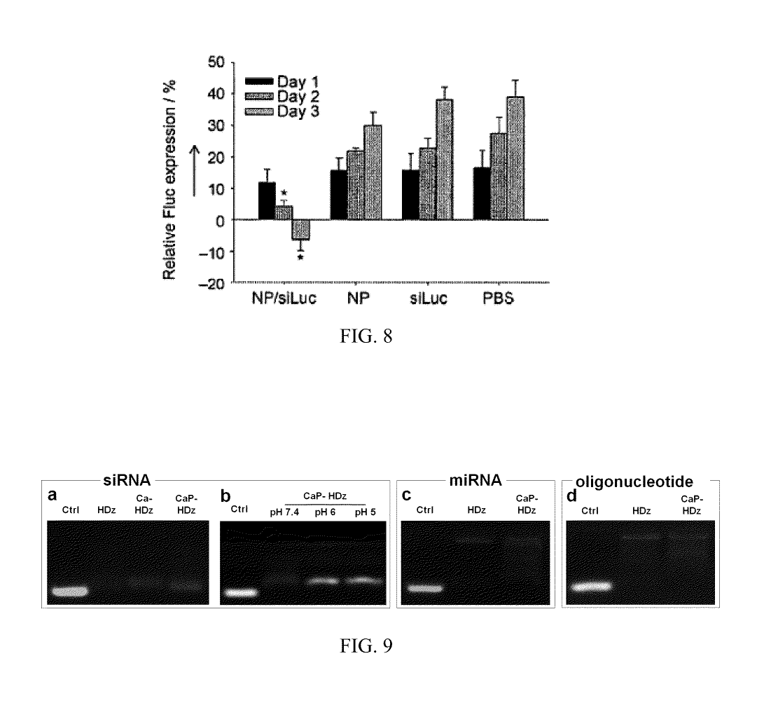

To demonstrate the use of the siLuc/HA_{DPA-Zn}-NPs in vivo, the gene silencing effect of siLuc/HA_{DPA-Zn}-NPs was investigated in a 4T1-fluc xenograft mouse model. Four groups of Balb/C mice (n=5 per group) with subcutaneous 4T1-fluc tumors were treated with an intratumor injection of 80 uL of a buffer solution that contained siLuc/HA_{DPA-Zn}-NPs (100 pmol/200 ug), HA_{DPA-Zn}-NPs (200 ug), naked siLuc (100 pmol), or phosphate-buffered saline (PBS). The silencing of the fluc gene was measured by in vivo BLI. FIG. 7 shows a series of typical optical images of fluc-expressing tumors after the administration of each formulation. After 72 hours, the level of fluc expression was significantly reduced in the tumor that was treated with siLuc/HA_{DPA-Zn}-NPs; however, the expression of fluc did not decrease in the other control groups. Quantitative analysis showed that the relative level of fluc expression was suppressed at 48 hours after the injection of siLuc/HA_{DPA-Zn}-NPs and was significantly inhibited to (−6.2±3.7)% at 72 hours. In contrast the control groups had increased levels of fluc expression ((30.0±4.1) %, (38.2±3.9)%, and (39.2±5.3)%, for HA_{DPA-Zn}-NPs, naked siRNA, and PBS, respectively, FIG. 8). The tumors from each group were harvested and weighed after BLI to determine whether the apparent gene silencing effect was a result of a reduction in the volume of the tumors as a consequence of the toxicity of the formulation. As shown by the in vitro testing, there was no significant difference in the neoplastic weights of the tumors between the various groups. This result implies that the downregulation of the fluc gene was selectively induced by the gene silencing mechanism.

### Example 8

This example illustrates the preparation and properties of further HA-based nano formulations for RNA therapeutics in accordance with an embodiment of the invention.

HA-based nanoparticles conjugated with an artificial RNA receptor, DPA/Zn were synthesized as previously reported. See Liu G. et al., “Sticky nanoparticles: a platform for siRNA delivery by a bis(zinc(II) dipicolylamine)-functionalized, self-assembled nanoconjugate.” Angew Chem Int Ed Engl. 2012 Jan. 9; 51(2):445-9.

Amine-functionalized bis(DPA) molecules and amphiphilic HA-CA (hyaluronic acid calcium salt) conjugates were first synthesized. To prepare HA-based nanoparticles (HA-NPs), HA-CA conjugates (60 mg) were dispersed in 12 mL of distilled water using a probe-type sonifier for 20 min. Then, bis(DPA) molecules (15 mg, 27 μmol), as an RNA receptor, were chemically conjugated onto HA-NPs through amide formation in the presence of EDC (1-Ethyl-3-(3-dimethylaminopropyl)carbodiimide) (10.5 mg, 54 μmol) and sulfo-NHS (sulfo-N-hydroxysuccinimide) (17.6 mg, 81 μmol). The HA-NPs modified with DPA molecules (HD-NPs) were purified by dialysis against distilled water for 24 h and were freeze-dried to get a dried white powder. To complex Zn ions with DPA ligands (HA_{DPA-Zn}-NPs), zinc nitrate hexahydrate (ZNH) (5 mL, 3 mg/mL, 10.1 mM) was mixed with HA_{DPA-Zn}-NPs (5 mL, 2 mg/mL in UPW), and the resulting mixture was incubated at 40° C. under agitation for 30 min. The final solution was purified with an Amicon ultra-15 centrifugal filter device (15 mL, 100 K MWCO) to remove unreacted Zn residues and was freeze-dried. The structure and purity of the HA_{DPA-Zn} conjugate were confirmed by ¹H-NMR and RP-HPLC, respectively.

To prepare Ca—P HA_{DPA-Zn}-NPs conjugated to nucleotides, HA_{DPA-Zn}-NP was dispersed in ultrapurified water (UPW) at 1 mg/mL by sonication using a probe-type sonifier for 20 min. The HA_{DPA-Zn}-NPs solution (2 μL) was vigorously mixed with 1 μL of RNAs, i.e., firefly luciferase-target siRNA (siLuc), microRNA-34a (miR-34a), or oligonucleotide 623 (Oligo-623) (see Table 1 below), and the solution was incubated at room temperature for 30 min. Inorganic calcium-phosphate (CaP) layers were deposited onto the nanoformulations by the in situ mineralization method to provide further protection of RNA molecules. 1 μL of Tris-calcium buffer (1 mM Tris, 250 m CaCl₂, pH 7.6) was added, and 4 μL of HEPES-phosphate buffer (50 mM HEPES, 1.5 mM Na₂HPO₄, pH 7.4) was sequentially added onto the HDz/siRNA nanoformulations. The solution was vigorously agitated by pipetting. The CaP-doped HA_{DPA-Zn}-NPs/RNA (CaP-HA_{DPA-Zn}-NPs/RNA) nanoformulations were treated for in vitro/in vivo application after having been incubated for 5 min at room temperature.

TABLE 1

| | |
|---|---|
| SEQUENCE ID | GCACUCUGAUUGACAAAUACGAUUU |
| NO. 1 | SiRNAi Firefly Luciferase, sense sequence 5′-3′ |
| SEQUENCE ID | AAAUCGUAUUUGUCAAUCAGAGUGC |
| NO. 2 | SiRNAi Firefly Luciferase, antisense sequence 5′-3′ |
| SEQUENCE ID | GGGCACAAGCUGGAGUACAACUACA |
| NO. 3 | SiRNAi GFP sense sequence 5′-3′ |
| SEQUENCE ID | UGUAGUUGUACUCCAGCUUGUGCCC |
| NO. 4 | SiRNAi GFP, antisense sequence 5′-3′ |
| SEQUENCE ID | AAUUCUCCGAACGUGUCACGU |
| NO. 5 | SiRNAi negative control, sense sequence 5′-3′ |
| SEQUENCE ID | ACGUGACACGUUCGGAGAAUU |
| NO. 6 | SiRNAi negative control, antisense sequence 5′-3′ |
| SEQUENCE ID | UGGCAGUGUCUUAGCUGGUUGUU |
| NO. 7 | miR34a, sense sequence 5′-3′ |
| SEQUENCE ID | AACAACCAGCUAAGACACUGCCA |
| NO. 8 | miR34a, antisense sequence 5′-3′ |
| SEQUENCE ID | UUGUACUACACAAAAGUACUG |
| NO. 9 | miRNA negative control, sense sequence 5′-3′ |
| SEQUENCE ID | CAGUACUUUUGUGUAGUACAA |
| NO. 10 | miRNA negative control, antisense sequence 5′-3′ |
| SEQUENCE ID | GTTATTCTTTAGAATGGTGC |
| NO. 11 | Oligo 623, sense sequence 5′-3′ |
| SEQUENCE ID | GTTATTCTTTAGAATGGTGC |
| NO. 12 | Oligo negative control, sense sequence 5′-3′ |

The CaP-HA_{DPA-Zn}-NPs/RNA formulations were tested for their ability to deliver nucleotides to cells, as described above. As shown in FIG. 9, siRNA, miRNA and oligonucleotide were securely complexed with HDz and CaP-HA_{DPA-Zn} at neutral pH (pH 7.4) (b). Rapid release of siRNA from CaP-HA_{DPA-Zn}-NP was observed in endosomal/lysosomal pH conditions (pH 6 and 5).

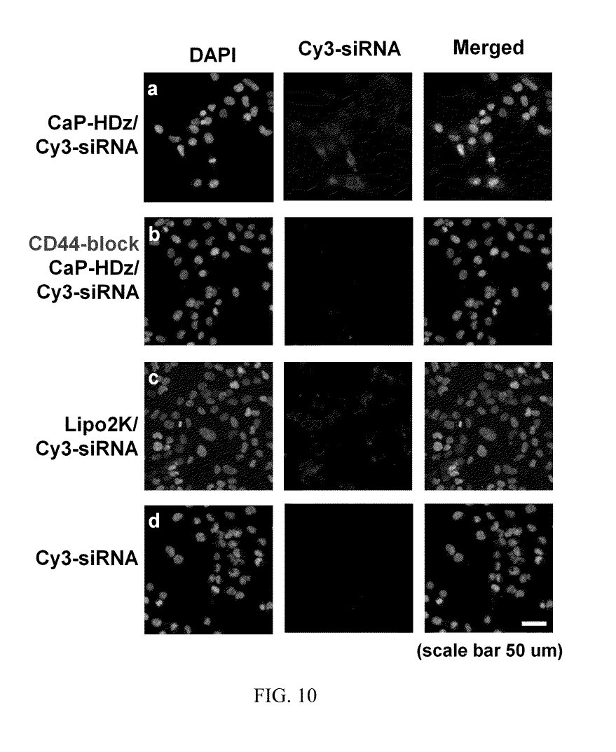

Cellular images of HCT116 cells treated with Cy3-siRNA complexed with CaP-HA_{DPA-Zn}-NP or Lipofectamine 2000 (Lipo2K) are shown FIG. 10 (a, b); CD44-blocked cells treated with CaP-HA_{DPA-Zn}/siRNA (c) or Cy3-siRNA only (d). The cells treated with CaP-HA_{DPA-Zn}/SiRNA exhibited significantly stronger fluorescence signal than the cells incubated with Lipo2K/siRNA. After CD44 receptors on the cell surface were blocked by pre-treatment of excess HA molecules, fluorescence signals from siRNA were rarely detected, indicating cell-permeation of CaP-HA_{DPA-Zn}/siRNA is highly dependant on the interaction between HA backbone of the NPs and CD44 cell surface receptors.

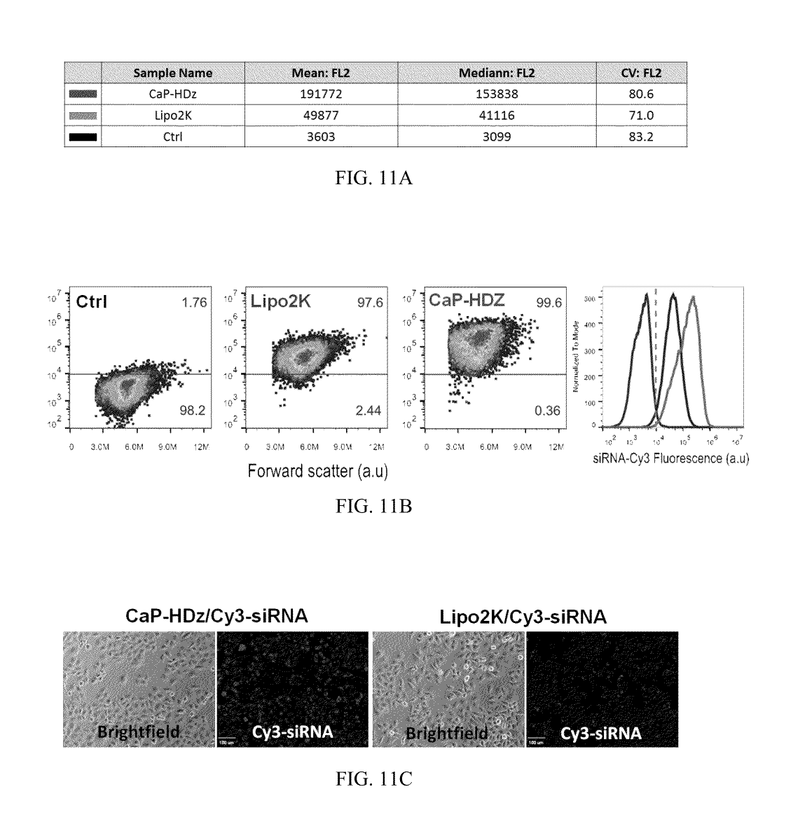

Quantitative FACS results and fluorescence images of DU145 cancer cells treated with CaP-HA_{DPA-Zn}/siRNA and Lipo2K/siRNA are shown in FIG. 11A-C. Mean fluorescence intensity and fluorescence signals of the NP-treated cells were remarkably higher than Lipo2K-treated cells.

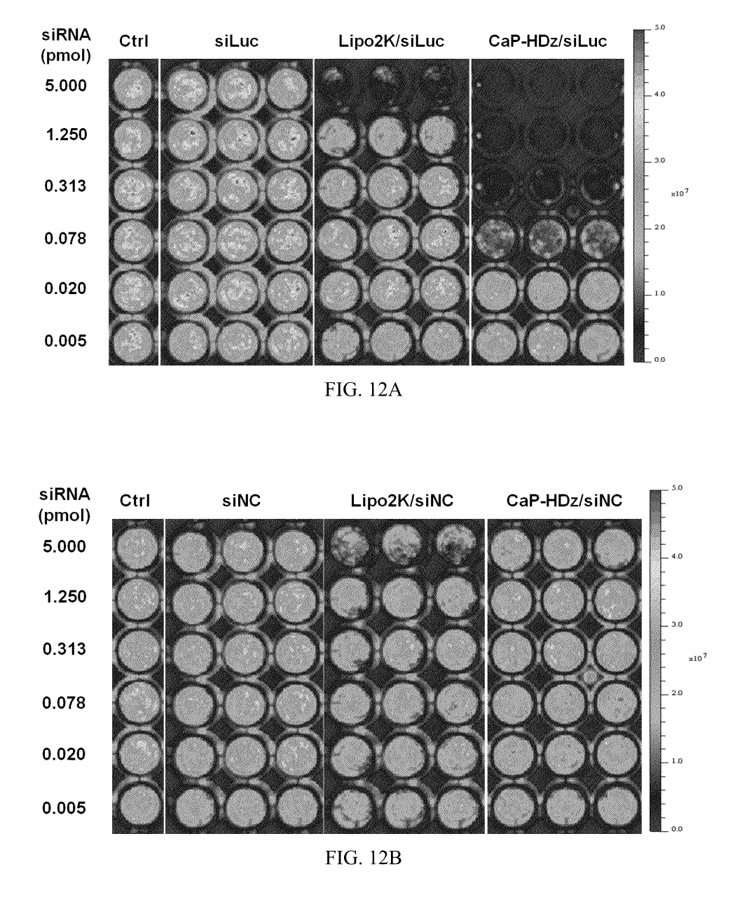

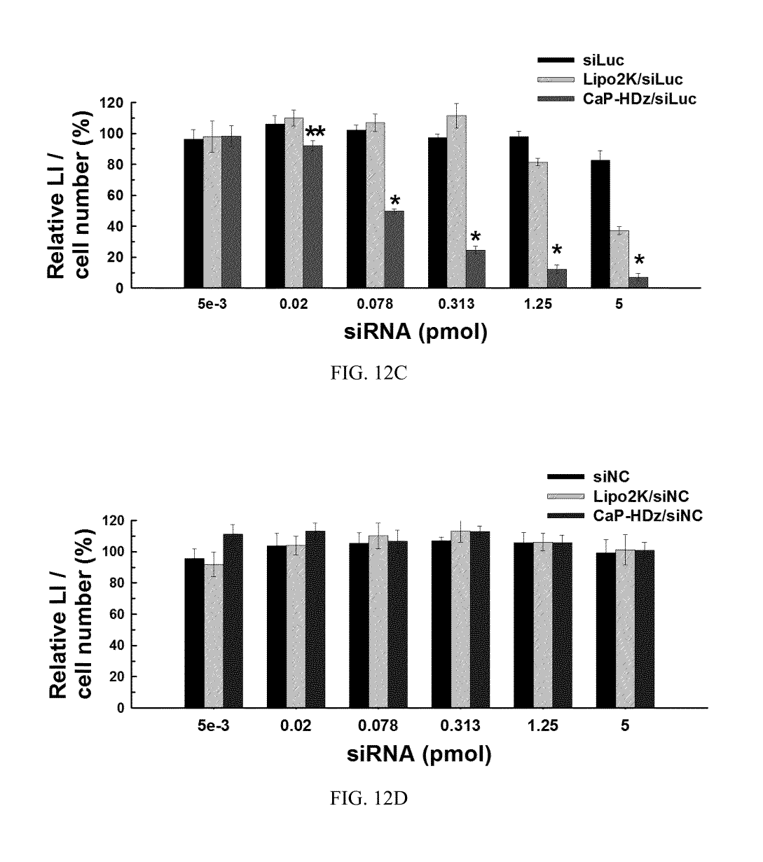

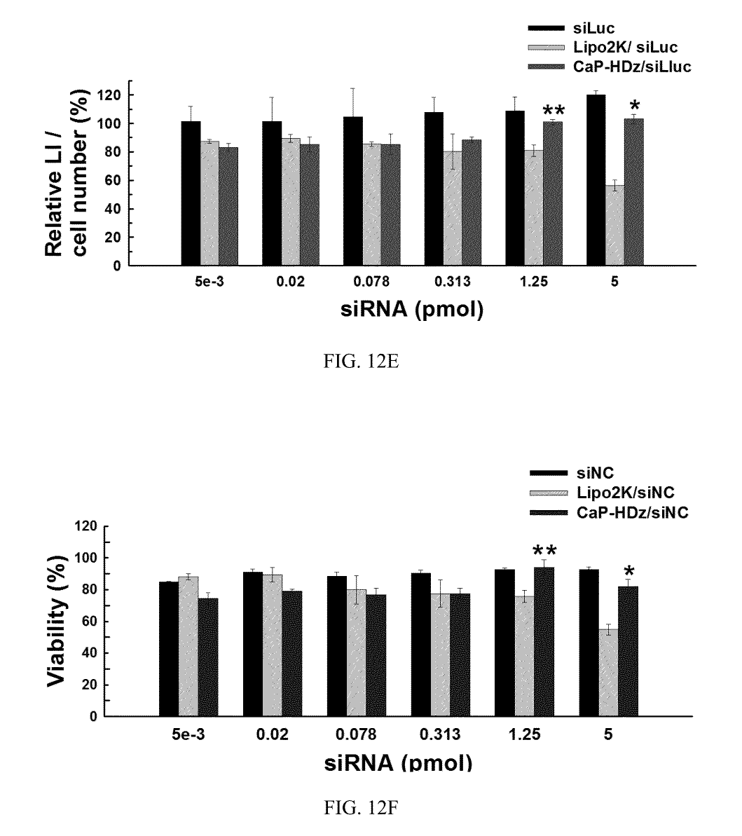

FIG. 12 depicts the suppression of fLuc gene expression and viability of 143B-fLuc cells after treatment with siRNA (siLuc or siNC) only, CaP-HA_{DPA-Zn}-NP or Lipo2K complexed with siLuc (FIG. 12A, FIG. 12C, FIG. 12E) or siNC (FIG. 12B, FIG. 12F). *p<0.005, **p<0.05 versus control or Lipo2K/siLuc. CaP-HA_{DPA-Zn}/siLuc group suppresses fLuc genes more efficiently than the Lipo2K/siLuc or free siLuc group. The CaP-HA_{DPA-Zn}-NP group also shows less toxicity compared to the Lipo2K group at high concentrations.

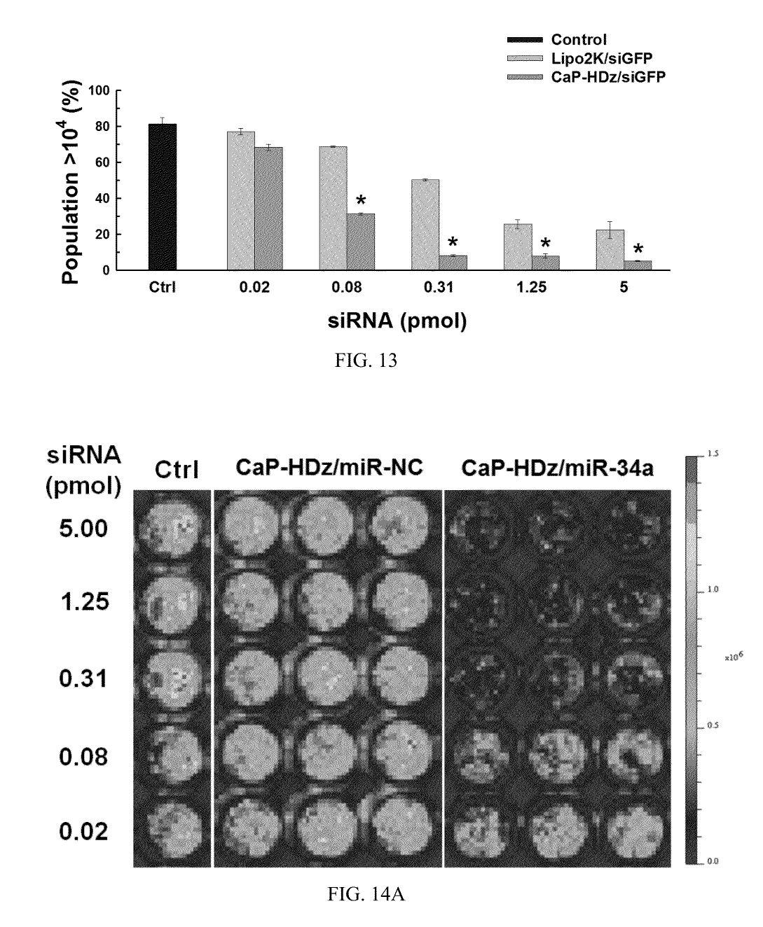

FIG. 13 depicts the suppression of GFP gene expression of DU145-GFP cells by CaP-HA_{DPA-Zn}-NP or Lipo2K complexed with siGFP. *p<0.005 versus Control group or Lipo2K/siGFP group. The CaP-HA_{DPA-Zn}/siGFP group shows suppression of the GFP gene expression more efficiently than the Lipo2K/siGFP group.

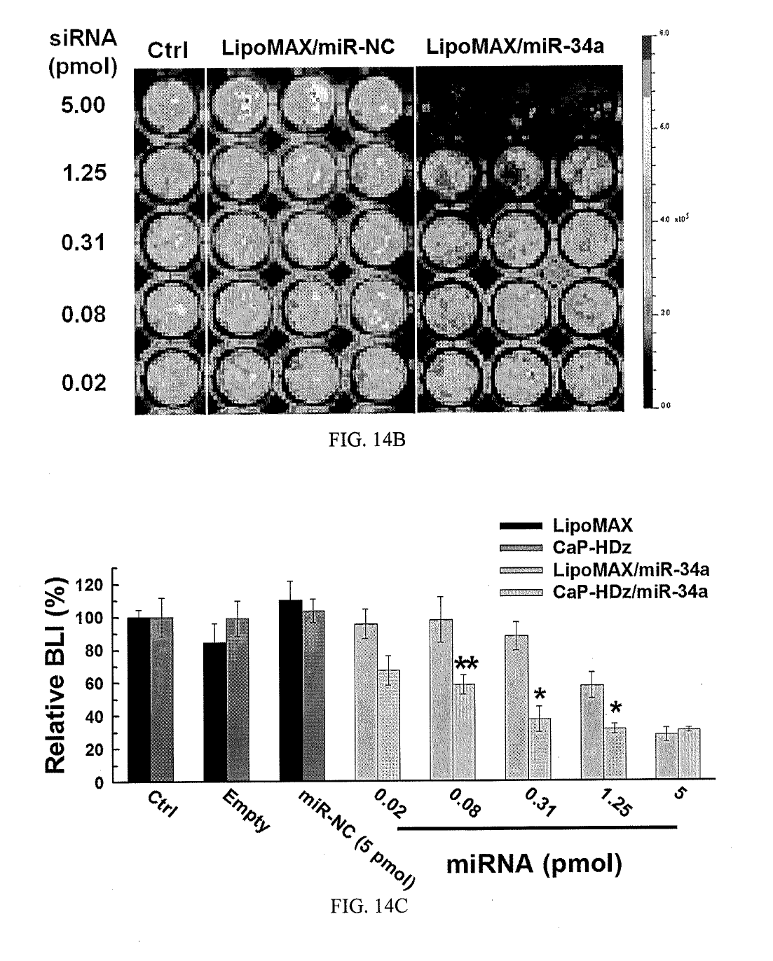

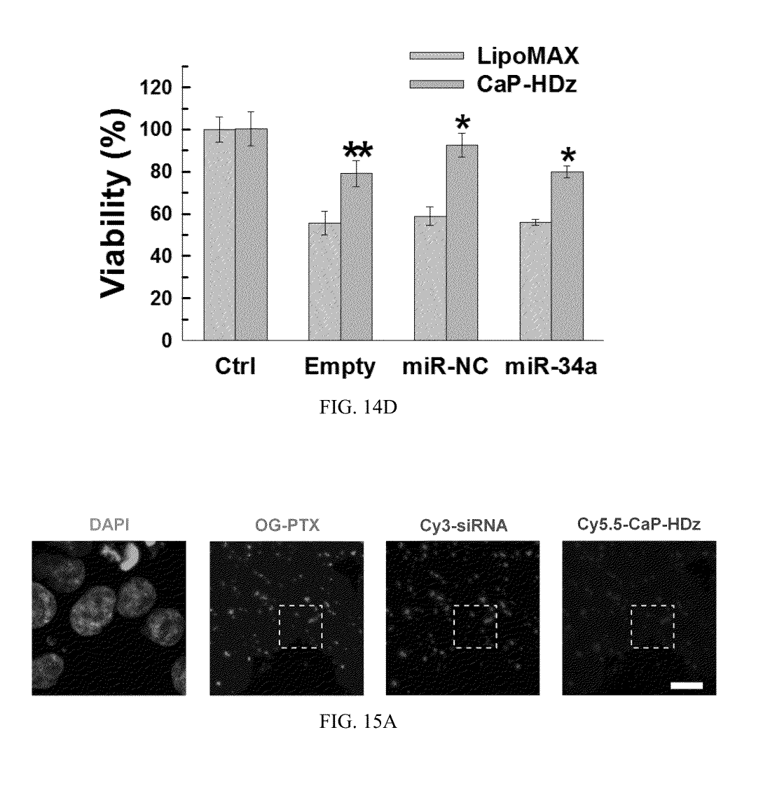

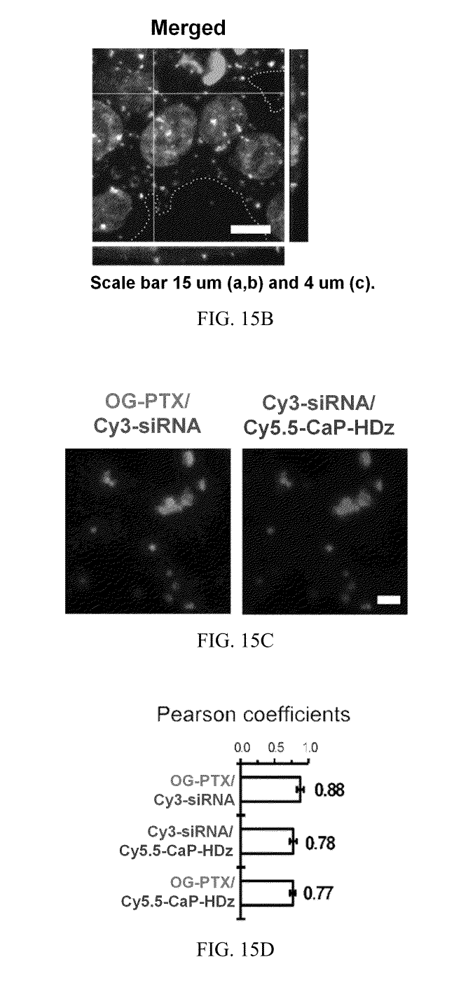

FIG. 14 depicts the suppression of fLuc signals in HCT116-fLuc-miR-34a cells by CaP-HA_{DPA-Zn}-NP (a,c) or LipoMAX (b, c) complexed with miR-34a, miR-NC or empty carriers without complexation. c depicts the viability of HCT116 cells after treatment of CaP-HA_{DPA-Zn} or LipoMAX complexed with miRNA (5 pmol) or empty carriers. *p<0.005, **p<0.05 versus the Control or LipoMAX/miRNA groups. CaP-HA_{DPA-Zn}/miR-34a group suppresses fLuc signals more efficiently than the LipoMAX/miR-34a group. The CaP-HA_{DPA-Zn}-NP group also shows less toxicity compared to the LipoMAX group at the high concentration (5 pmol).

FIG. 15, a, depicts the multi-channel fluorescence images of DU145 cells treated with Cy5.5-labeled CaP-HA_{DPA-Zn}-NP complexed with Cy3-siRNA and loaded with Oregon green-conjugated paclitaxel (OG-PTX). b depicts the merged images depicted in a. c and d depict cellular images and co-localization efficiency of PTX/siRNA, siRNA/CaP-HA_{DPA-Zn} or PTX/CaP-HA_{DPA-Zn}. Considerable amount of CaP-HDz-NP was internalized into the DU145 cells along with miRNA and PTX. The three major components were co-localized in the cells at early time points (30 min after the treatment).

The invention includes the following aspects:

1. A nanoparticulate complex comprising an artificial phosphate receptor of formula (I):

P-[L-[-N(CH₂-2-pyridyl)₂]]ₚ.pZn²⁺ (I)

wherein P represents a nanoparticulate substrate,

L represents a linking group, and

p is an integer of ≧1,

in combination with an anion or anions.

2. The complex of aspect 1, wherein the nanoparticulate substrate is a synthetic organic polymeric substrate, a biopolymeric substrate, or an inorganic substrate.

3. The complex of aspect 1 or 2, wherein the nanoparticulate substrate comprises a polysaccharide.

4. The complex of any one of aspects 1-3, wherein the nanoparticulate substrate comprises hyaluronic acid.

5. The complex of any one of aspects 1-4, wherein the nanoparticulate substrate comprises the structure:

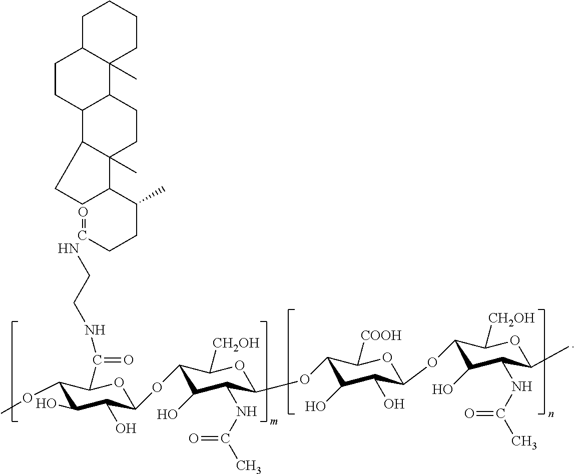

6. The complex of any one of aspects 1-5, wherein L comprises a substituted or unsubstituted aryl group.

7. The complex of any one of aspects 1-6, wherein L comprises an Ω-(3,5-disubstituted aryl)alkylamino group.

8. The complex of aspect 7, wherein L-[-N(CH₂-2-pyridyl)₂] is:

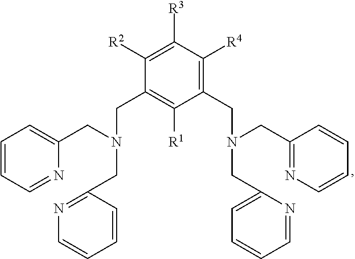

wherein R¹ is hydrogen or —OH,

wherein R² and R⁴ are independently hydrogen or C₁-C₆ alkyl, and

wherein R³ is —NH-alkyl.

9. The complex of aspect 8, wherein L-[-N(CH₂-2-pyridyl)₂] is:

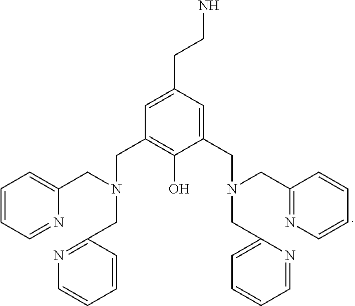 [SMILES: NCCc1cc(CN(Cc2ccccn2)Cc2ccccn2)c(O)c(CN(Cc2ccccn2)Cc2ccccn2)c1]

10. The complex of any one of aspects 1-9, wherein the artificial phosphate receptor of formula (I) is:

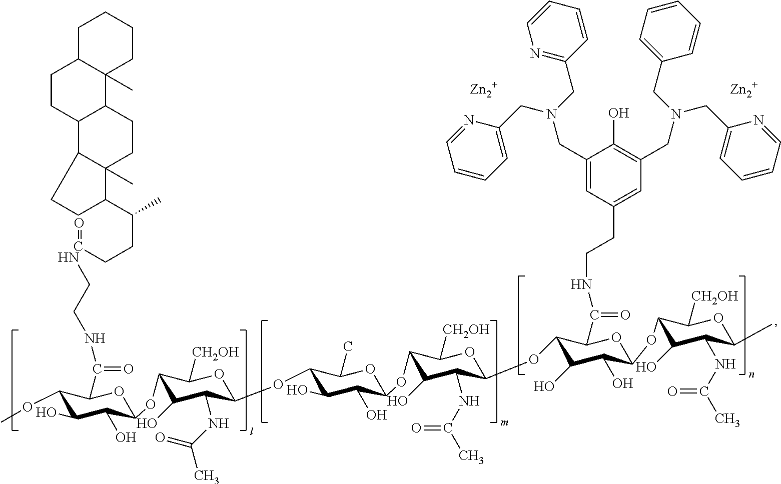

wherein l, m, and n are independently integers of from 1 to about 10,000.

11. The complex of aspect 10, wherein the ratio of n to (1+m) is from 0.01 to about 1.0.

12. The complex of aspect 11, wherein the ratio of n to (1+m) is from 0.1 to about 0.5.

13. A phosphate anion ligand complex comprising at least one phosphate anion ligand complexed with the nanoparticulate complex of any one of aspects 1-12.

14. The phosphate anion ligand complex of aspect 13, wherein the phosphate anion ligand is selected from siRNA, miRNA, oligonucleotides, RNA, and DNA.

15. The phosphate anion ligand complex of aspect 13, wherein the phosphate anion ligand is siRNA.

16. An anticancer complex comprising an anticancer agent and the phosphate anion ligand complex of any one of aspects 13-15.

17. A pharmaceutical composition comprising the complex of any one of aspects 13-15 and a pharmaceutically acceptable carrier.

18. A pharmaceutical composition comprising the anticancer complex of aspect 16 and a pharmaceutically acceptable carrier.

19. A method for silencing a gene in a cancer patient in need thereof comprising administering an effective amount of the complex of any one of aspects 13-15, the anticancer complex of aspect 16, or the pharmaceutical composition of aspects 17 or 18.

20. A method for treating or preventing cancer in a patient in need thereof, comprising administering an effective amount of the complex of any one of aspects 13-15, the anticancer complex of aspect 16, or the pharmaceutical composition of aspects 17 or 18 to the patient.

21. A method for targeting a cell in cancer treatment, comprising contacting the cell with the complex of any one of aspects 13-15, the anticancer complex of aspect 16, or the pharmaceutical composition of aspects 17 or 18.

22. A nanoparticulate complex comprising an artificial phosphate receptor of formula (I):

P-[L-[-N(CH₂-2-pyridyl)₂]]ₚ.pZn²⁺ (I)

wherein P represents a nanoparticulate substrate,

L represents a linking group, and

p is an integer of ≧1,

for use in treating cancer.

21. A kit comprising a nanoparticulate complex comprising an artificial phosphate receptor of formula (I):

P-[L-[-N(CH₂-2-pyridyl)₂]]ₚ.pZn²⁺ (I)

wherein P represents a nanoparticulate substrate,

L represents a linking group, and

p is an integer of ≧1,

at least one nucleic acid, and optionally at least one anticancer agent, and instructions for use thereof.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A nanoparticulate complex comprising an artificial phosphate receptor of formula (I):
P-[L-[-N(CH₂-2-pyridyl)₂]]ₚ.pZn²⁺ (I)
wherein P represents a nanoparticulate substrate,
L represents a linking group, and
p is an integer of ≧1,
in combination with an anion or anions.

2. The complex of claim 1, wherein the nanoparticulate substrate is a synthetic organic polymeric substrate, a biopolymeric substrate, or an inorganic substrate.

3. The complex of claim 1, wherein the nanoparticulate substrate comprises a polysaccharide.

4. The complex of claim 2, wherein the nanoparticulate substrate comprises hyaluronic acid.

5. The complex of claim 4, wherein the nanoparticulate substrate comprises the structure:

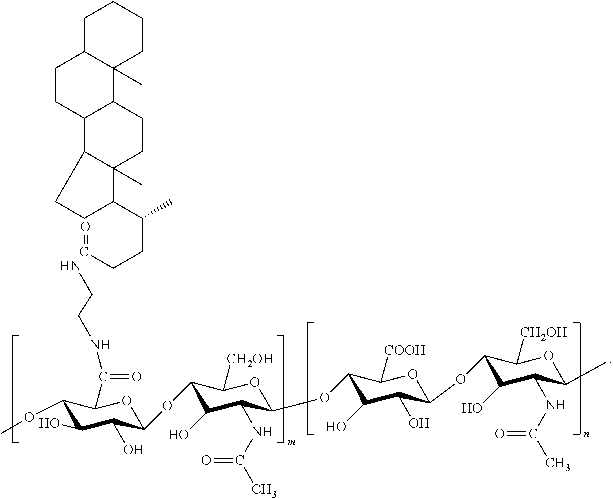

6. The complex of claim 1, wherein L comprises a substituted or unsubstituted aryl group.

7. The complex of claim 6, wherein L comprises an Ω-(3,5-disubstituted aryl)alkylamino group.

8. The complex of claim 7, wherein L-[-N(CH₂-2-pyridyl)₂] is: 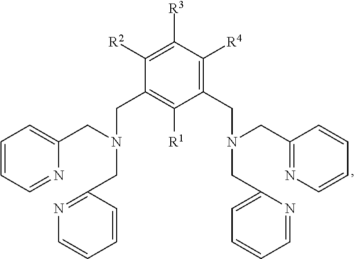
wherein R¹ is hydrogen or —OH,
wherein R² and R⁴ are independently hydrogen or C₁-C₆ alkyl, and
wherein R³ is —NH-alkyl.

9. The complex of claim 8, wherein L-[-N(CH₂-2-pyridyl)₂] is:

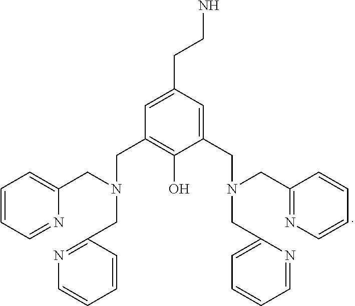 [SMILES: NCCc1cc(CN(Cc2ccccn2)Cc2ccccn2)c(O)c(CN(Cc2ccccn2)Cc2ccccn2)c1]

10. The complex of claim 9, wherein the artificial phosphate receptor of formula (I) is: 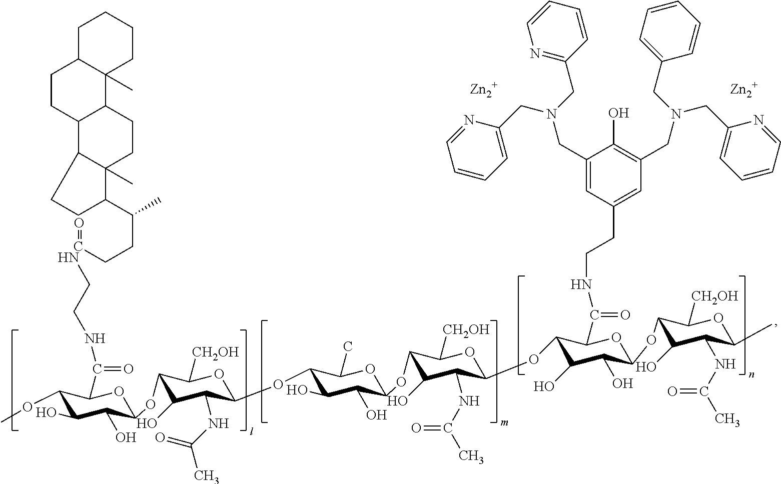
wherein l, m, and n are independently integers of from 1 to about 10,000.

11. The complex of claim 10, wherein the ratio of n to (1+m) is from 0.01 to about 1.0.

12. The complex of claim 11, wherein the ratio of n to (1+m) is from 0.1 to about 0.5.

13. A phosphate anion ligand complex comprising at least one phosphate anion ligand complexed with the nanoparticulate complex of claim 1.

14. The phosphate anion ligand complex of claim 13, wherein the phosphate anion ligand is selected from siRNA, miRNA, oligonucleotides, RNA, and DNA.

15. The phosphate anion ligand complex of claim 13, wherein the phosphate anion ligand is siRNA.

16. An anticancer complex comprising an anticancer agent and the phosphate anion ligand complex of claim 13.

17. A pharmaceutical composition comprising the complex of claim 13 and a pharmaceutically acceptable carrier.

18. A pharmaceutical composition comprising the anticancer complex of claim 16 and a pharmaceutically acceptable carrier.

19. A method for silencing a gene in a cancer patient in need thereof comprising administering an effective amount of the complex of claim 13.

20. A method for silencing a gene in a cancer patient in need thereof comprising administering an effective amount of the anticancer complex of claim 16.

21. A method for silencing a gene in a cancer patient in need thereof comprising administering an effective amount of the pharmaceutical composition of claim 17.

22. A method for silencing a gene in a cancer patient in need thereof comprising administering an effective amount of the pharmaceutical composition of claim 18.

23. A method for treating or preventing cancer in a patient in need thereof, comprising administering an effective amount of the complex of claim 13.

24. A method for treating or preventing cancer in a patient in need thereof, comprising administering an effective amount of the anticancer complex of claim 16.

25. A method for treating or preventing cancer in a patient in need thereof, comprising administering an effective amount of the pharmaceutical composition of claim 17 to the patient.

26. A method for treating or preventing cancer in a patient in need thereof, comprising administering an effective amount of the pharmaceutical composition of claim 18 to the patient.

27. A method for targeting a cell in cancer treatment, comprising contacting the cell with the complex of claim 13.

28. A method for targeting a cell in cancer treatment, comprising contacting the cell with the anticancer complex of claim 16.

29. A method for targeting a cell in cancer treatment, comprising contacting the cell with the pharmaceutical composition of claim 17.

30. A method for targeting a cell in cancer treatment, comprising contacting the cell with the pharmaceutical composition of claim 18.

31. A nanoparticulate complex comprising an artificial phosphate receptor of formula (I):
P-[L-[-N(CH₂-2-pyridyl)₂]]ₚ.pZn²⁺ (I)
wherein P represents a nanoparticulate substrate,
L represents a linking group, and
p is an integer of ≧1,
for use in treating cancer.

32. A kit comprising a nanoparticulate complex comprising an artificial phosphate receptor of formula (I):
P-[L-[-N(CH₂-2-pyridyl)₂]]ₚ.pZn²⁺ (I)
wherein P represents a nanoparticulate substrate,
L represents a linking group, and
p is an integer of ≧1,
at least one nucleic acid, and optionally at least one anticancer agent, and instructions for use thereof.


SEQUENCE LISTING (12 sequences)

SEQ ID NO: 1 - RNA, 25 nt, Artificial Sequence
  Synthetic
gcacucugauugacaaauacgauuu

SEQ ID NO: 2 - RNA, 25 nt, Artificial Sequence
  Synthetic
aaaucguauuugucaaucagagugc

SEQ ID NO: 3 - RNA, 25 nt, Artificial Sequence
  Synthetic
gggcacaagcuggaguacaacuaca

SEQ ID NO: 4 - RNA, 25 nt, Artificial Sequence
  Synthetic
uguaguuguacuccagcuugugccc

SEQ ID NO: 5 - RNA, 21 nt, Artificial Sequence
  Synthetic
aauucuccgaacgugucacgu

SEQ ID NO: 6 - RNA, 21 nt, Artificial Sequence
  Synthetic
acgugacacguucggagaauu

SEQ ID NO: 7 - RNA, 23 nt, Artificial Sequence
  Synthetic
uggcagugucuuagcugguuguu

SEQ ID NO: 8 - RNA, 23 nt, Artificial Sequence
  Synthetic
aacaaccagcuaagacacugcca

SEQ ID NO: 9 - RNA, 21 nt, Artificial Sequence
  Synthetic
uuguacuacacaaaaguacug

SEQ ID NO: 10 - RNA, 21 nt, Artificial Sequence
  Synthetic
caguacuuuuguguaguacaa

SEQ ID NO: 11 - DNA, 20 bp, Artificial Sequence
  Synthetic
gttattctttagaatggtgc

SEQ ID NO: 12 - DNA, 20 bp, Artificial Sequence
  Synthetic
gtaattatttataatcgtcc